(19) United States
(12) Patent Application Publication
(10) Pub. No.: US 2024/0299712 A1
(43) Pub. Date: Sep. 12, 2024

(54) SUBINTIMAL RE-ENTRY CATHETER WITH INTRAVASCULAR ULTRASOUND CAPABILITIES

(71) Applicant: Boston Scientific Scimed, Inc., Maple Grove, MN (US)

(72) Inventors: Gordon John Kocur, Lino Lakes, MN (US); Jeffrey Steven Fuller, Brooklyn Park, MN (US); Jeffrey Adam Hammerstrom, New Hope, MN (US); Kyle Logan Lemke, Hudson, WI (US)

(73) Assignee: Boston Scientific Scimed, Inc., Maple Grove, MN (US)

(21) Appl. No.: 18/598,443

(22) Filed: Mar. 7, 2024

Related U.S. Application Data
(60) Provisional application No. 63/451,372, filed on Mar. 10, 2023.

(51) Int. Cl. A61M 25/10; A61M 25/00

(52) U.S. Cl. CPC A61M 25/1011; A61M 25/0023; A61M 2025/1061; A61M 2209/00

(57) ABSTRACT

A recanalization catheter for facilitating reentry into a lumen of a vessel having an occlusion therein from a subintimal space in a wall of the vessel. The recanalization catheter includes a catheter shaft extending distally from a hub. The catheter shaft defines a guidewire lumen extending therethrough. First and second inflatable balloon members are disposed on a distal end region of the catheter shaft. The catheter shaft includes an inflation lumen in fluid communication with the first and second inflatable balloon members. A lateral opening which is in communication with the guidewire lumen opens to an exterior of the catheter shaft between the first and second inflatable balloon members on a first side of the catheter shaft. The catheter shaft also includes an imaging lumen extending through the catheter shaft configured to receive an IVUS imaging device therein.

### CROSS-REFERENCE TO RELATED APPLICATIONS

This application claims the benefit of U.S. Provisional Patent Application Ser. No. 63/451,372, filed Mar. 10, 2023, which is incorporated herein by reference.

### TECHNICAL FIELD

The disclosure is directed to devices and methods for recanalization of an occluded blood vessel. More particularly, the disclosure is directed to devices and methods for re-entry into the true lumen from the extraluminal or subintimal space of a blood vessel.

### BACKGROUND

Chronic total occlusion (CTO) is an arterial vessel blockage that obstructs blood flow through the vessel, and can occur in both coronary and peripheral arteries. In some instances, it may be difficult or impossible to pass through the CTO with a medical device in an antegrade direction to recanalize the vessel. Accordingly, techniques have been developed for creating a subintimal pathway (i.e., a pathway between the intimal and adventitial tissue layers of the vessel) around the occlusion and then re-entering the true lumen of the vessel distal of the occlusion in an attempt to recanalize the vessel. In some instances, re-entering the true lumen from the subintimal space and/or recanalization can be difficult using only angiography. Accordingly, it is desirable to provide alternative recanalization devices with integrated intravascular ultrasound (IVUS) to enable live imaging during subintimal advancement and re-entry and/or associated methods of recanalizing a blood vessel in which a CTO is present.

### SUMMARY

The disclosure is directed to several alternative designs, materials and methods of manufacturing medical device structures and assemblies, and uses thereof.

Accordingly, one illustrative example is a recanalization catheter for facilitating reentry into a lumen of a vessel having an occlusion therein from a subintimal space in a wall of the vessel. The recanalization catheter includes a catheter shaft extending distally from a hub. The catheter shaft defines a guidewire lumen extending therethrough. First and second inflatable balloon members are disposed on a distal end region of the catheter shaft. The catheter shaft includes an inflation lumen in fluid communication with the first and second inflatable balloon members. A lateral opening, which is in communication with the guidewire lumen, opens to an exterior of the catheter shaft between the first and second inflatable balloon members on a first side of the catheter shaft. The catheter shaft also includes an imaging lumen extending through the catheter shaft configured to receive an IVUS imaging device therein.

In addition or alternatively to any example disclosed herein, the inflation lumen includes a first inflation lumen in fluid communication with the first inflatable balloon member and a second inflation lumen in fluid communication with the second inflatable balloon member.

In addition or alternatively to any example disclosed herein, in a proximal portion of the catheter shaft a central longitudinal axis of the catheter shaft passes through the imaging lumen, and an entirety of each of the first inflation lumen, the second inflation lumen, and the guidewire lumen is positioned on a first side of a first imaginary plane extending parallel to and passing through the central longitudinal axis.

In addition or alternatively to any example disclosed herein, in a distal portion of the catheter shaft, the imaging lumen and the guidewire lumen are positioned between the first and second inflation lumens such that a second imaginary plane extending parallel to and passing through the central longitudinal axis passes through each of the first inflation lumen, the second inflation lumen, the imaging lumen, and the guidewire lumen.

In addition or alternatively to any example disclosed herein, a proximal portion of the catheter shaft has a circular cross-sectional shape and a distal portion of the catheter shaft has a generally rectangular cross-sectional shape.

In addition or alternatively to any example disclosed herein, the first and second inflatable balloon member are configured to be inflated laterally from opposite sides of the catheter shaft along a third imaginary plane, wherein the third imaginary plane passes through each of the imaging lumen and the guidewire lumen.

In addition or alternatively to any example disclosed herein, the recanalization catheter further includes an IVUS imaging device positionable within the imaging lumen to position an IVUS transducer distal of the first lateral opening.

In addition or alternatively to any example disclosed herein, an outer diameter of the catheter shaft is 0.059 inches or less, the guidewire lumen has a diameter of about 0.017 inches, and a minimum distance across the imaging lumen through a center axis of the imaging lumen is 0.033 inches or more.

In addition or alternatively to any example disclosed herein, an outer diameter of the catheter shaft is 0.052 inches or less, the guidewire lumen has a diameter of about 0.017 inches, and a minimum distance across the imaging lumen through a center axis of the imaging lumen is 0.026 inches or more.

In addition or alternatively to any example disclosed herein, a wall thickness of the catheter shaft at all locations along the catheter shaft is at least 0.003 inches.

Another example is a recanalization catheter for facilitating reentry into a lumen of a vessel having an occlusion therein from a subintimal space in a wall of the vessel. The recanalization catheter includes a catheter shaft extending distally from a hub and an expandable balloon disposed on a distal end region of the catheter shaft. The catheter shaft is an extruded tubular member defining a guidewire lumen extending therethrough, an inflation lumen in fluid communication with an interior of the expandable balloon, and an imaging lumen extending through the catheter shaft configured to receive an IVUS imaging device therein.

In addition or alternatively to any example disclosed herein, the recanalization catheter further includes an IVUS imaging device positional within the imaging lumen such that an IVUS transducer of the IVUS imaging device is located distal of the expandable balloon.

In addition or alternatively to any example disclosed herein, the recanalization catheter further includes a lateral opening in communication with the guidewire lumen. The lateral opening opens to an exterior of the catheter shaft between a proximal end of the expandable balloon and a distal end of the expandable balloon.

In addition or alternatively to any example disclosed herein, the inflation lumen includes a first inflation lumen in fluid communication with a first inflatable balloon member of the expandable balloon and a second inflation lumen in fluid communication with a second inflatable balloon member of the expandable balloon. In a proximal portion of the catheter shaft, a central longitudinal axis of the catheter shaft passes through the imaging lumen, and an entirety of each of the first inflation lumen, the second inflation lumen, and the guidewire lumen is positioned on a first side of a first imaginary plane extending parallel to and passing through the central longitudinal axis.

In addition or alternatively to any example disclosed herein, in a distal portion of the catheter shaft, the imaging lumen and the guidewire lumen are positioned between the first and second inflation lumens such that a second imaginary plane extending parallel to and passing through the central longitudinal axis passes through each of the first inflation lumen, the second inflation lumen, the imaging lumen, and the guidewire lumen. Yet another example is a recanalization catheter for facilitating reentry into a lumen of a vessel having an occlusion therein from a subintimal space in a wall of the vessel. The recanalization catheter includes a catheter shaft extending distally from a hub and an expandable balloon disposed on a distal end region of the catheter shaft, the expandable balloon including a first inflatable balloon member on a first lateral side of the catheter shaft and a second inflatable balloon member on a second lateral side of the catheter shaft opposite the first lateral side. The catheter shaft includes an extruded proximal tubular member having a circular cross-sectional shape and an extruded distal tubular member having a generally rectangular cross-sectional shape. The catheter shaft defines a guidewire lumen extending through the extruded proximal tubular member and the extruded distal tubular member, a first inflation lumen extending through the extruded proximal tubular member and the extruded distal tubular member, a second inflation lumen extending through the extruded proximal tubular member and the extruded distal tubular member, and an imaging lumen extending through the extruded proximal tubular member and the extruded distal tubular member. The first inflation lumen is in fluid communication with an interior of the first inflatable balloon member and the second inflation lumen is in fluid communication with an interior of the second inflatable balloon member. The imaging lumen is configured to receive an IVUS imaging device therein.

In addition or alternatively to any example disclosed herein, the recanalization catheter further includes a lateral opening in communication with the guidewire lumen. The lateral opening opens to an exterior of the catheter shaft between the first and second inflatable balloon members.

In addition or alternatively to any example disclosed herein, the recanalization catheter further includes an IVUS imaging device positional within the imaging lumen such that an IVUS transducer of the IVUS imaging device is located distal of the lateral opening, preferably distal of the expandable balloon.

In addition or alternatively to any example disclosed herein, throughout the extruded proximal tubular member a central longitudinal axis of the catheter shaft passes through the imaging lumen, and an entirety of each of the first inflation lumen, the second inflation lumen, and the guidewire lumen is positioned on a first side of a first imaginary plane extending parallel to and passing through the central longitudinal axis. In addition or alternatively to any example disclosed herein, throughout the extruded distal tubular member, the imaging lumen and the guidewire lumen are positioned between the first and second inflation lumens such that a second imaginary plane extending parallel to and passing through the central longitudinal axis passes through each of the first inflation lumen, the second inflation lumen, the imaging lumen, and the guidewire lumen. Yet another example is a recanalization catheter for facilitating reentry into a lumen of a vessel having an occlusion therein from a subintimal space in a wall of the vessel. The catheter may include an elongate catheter shaft having a guidewire lumen and an imaging lumen extending therein. The distal end region of the catheter shaft may include a lateral opening in communication with the guidewire lumen. The catheter shaft may include a reinforcement structure, such as a braid or coil, to facilitate torque transmission of the catheter shaft such that the lateral opening may be rotationally oriented toward the true lumen of the blood vessel distal of the occlusion. A reentry device may be advanced through the guidewire lumen and out the lateral opening to reenter the true lumen distal of the occlusion. The imaging lumen is configured to receive an IVUS imaging device therein. The IVUS imaging device is configured to provide an ultrasound image of the blood vessel to confirm the rotational orientation of the lateral opening in the subintimal space and/or the trajectory of the re-entry device from the lateral opening toward the true lumen.

The above summary of some example embodiments is not intended to describe each disclosed embodiment or every implementation of the aspects of the disclosure.

### BRIEF DESCRIPTION OF THE DRAWINGS

The aspects of the disclosure may be more completely understood in consideration of the following detailed description of various embodiments in connection with the accompanying drawings, in which:

FIG. 1 is a perspective view of an exemplary catheter for recanalization of a blood vessel;

FIG. 2A is a cross-sectional view of the catheter shaft of the catheter of FIG. 1 taken along line 2-2;

FIG. 2B is an alternative cross-sectional view of the catheter shaft of the catheter of FIG. 1 taken along line 2-2;

FIG. 2C is an alternative cross-sectional view of the catheter shaft of the catheter of FIG. 1 taken along line 2-2;

FIG. 2D is an alternative cross-sectional view of the catheter shaft of the catheter of FIG. 1 taken along line 2-2;

FIG. 3 is a cross-sectional view of the catheter shaft of the catheter of FIG. 1 taken along line 3-3;

FIG. 4 is a cross-sectional view of the catheter shaft of the catheter of FIG. 1 taken along line 4-4;

FIG. 5 is a longitudinal cross-sectional view of the distal end region of the catheter of FIG. 1;

FIGS. 6-10 illustrate aspects of an exemplary method for recanalizing an occluded blood vessel using the catheter of FIG. 1.

FIG. 11 is a plan view of an exemplary catheter for recanalization of a blood vessel;

FIG. 12 is a cross-sectional view of the catheter shaft of the catheter of FIG. 11, taken along line 12-12 of FIG. 11.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

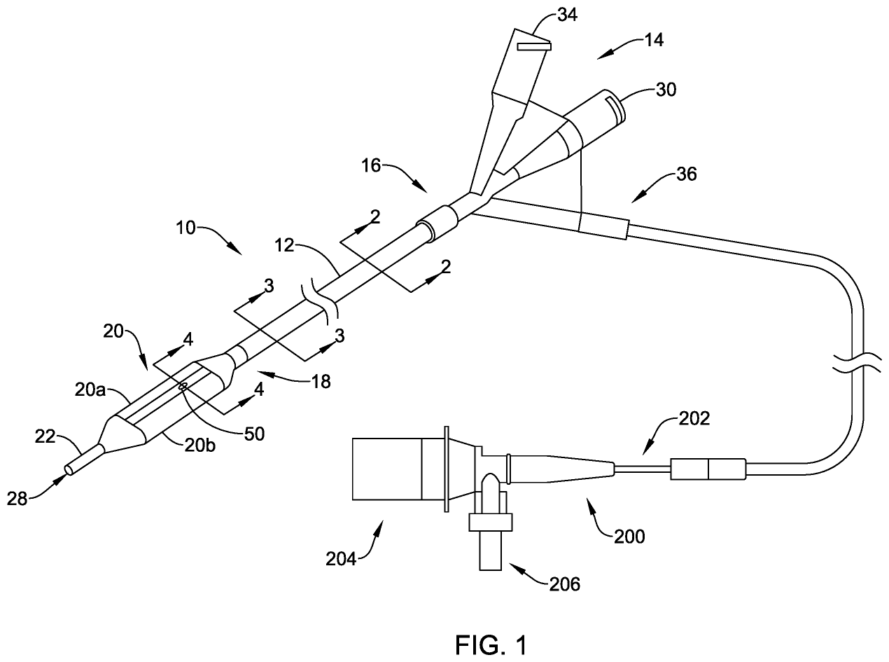

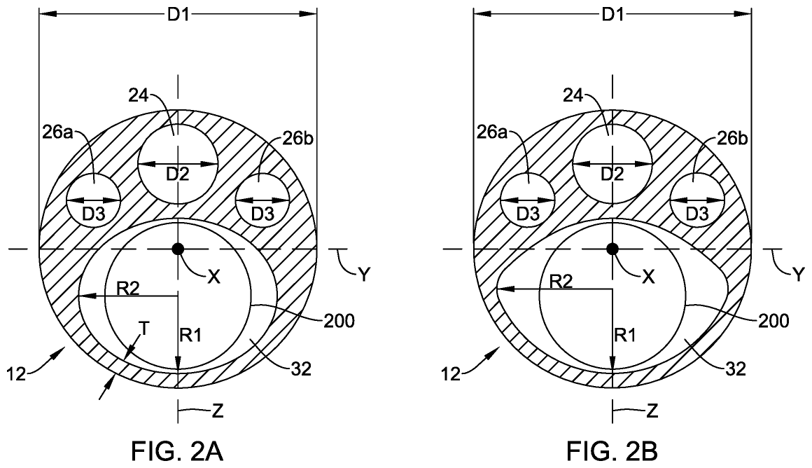

An exemplary recanalization catheter 10 is illustrated at FIG. 1. The recanalization catheter 10 may include a catheter shaft 12 extending from a hub assembly 14 at a proximal end 16 of the catheter shaft 12 to an expandable balloon 20 mounted on a distal end region 18 of the catheter shaft 12. The distal end region 18 of the catheter shaft 12 may extend through the expandable balloon 20 to form a distal tip 22 extending distal of the expandable balloon 20. As will be described further herein, in some instances the expandable balloon 20 may be formed of one or more, or a plurality of inflatable balloon members 20a, 20b which may be inflated with an inflation fluid delivered through an inflation lumen 26 (Sec FIGS. 2A-2D) of the catheter shaft 12, as described further herein.

The catheter 10 may be configured to be advanced over a guidewire for delivery to a remote location in the vasculature of a patient. For example, in some instances the catheter 10 may be configured as an over-the-wire (OTW) catheter having a guidewire lumen 24 (see FIGS. 2A-2D) extending through the entire length of the catheter 10 from a distal opening 28 at the distal tip 22 of the catheter 10 to a proximal guidewire port 30 in the hub assembly 14. The distal opening 28 may be located at a distalmost extent of the catheter 10. In other instances, the catheter 10 may be configured as a single-operator-exchange (SOE) catheter having a guidewire lumen 24 extending from the distal opening 28 to a proximal guidewire port (not shown) located a short distance proximal of the expandable balloon 20 and distal of the hub assembly 14. In such a configuration, the guidewire may extend through the guidewire lumen 24 between the distal opening 28 and the proximal guidewire port, and extend along an exterior of the catheter shaft 12 proximal of the proximal guidewire port to the proximal end 16 of the catheter shaft 12. It is noted that in instances in which the catheter 10 is an SOE catheter, the hub assembly 14 may not include a proximal guidewire port 30.

The recanalization catheter 10 may include and/or accommodate an intravascular ultrasound (IVUS) imaging device 200. The IVUS imaging device 200 may include an elongate shaft 202 extending distally from a hub 202. The IVUS imaging device 200 may also include a flush port 206 in some instances. The IVUS imaging device 200 may be coupled to, extend from, and/or be received by the port 36 such that the elongate shaft 202 of the IVUS imaging device 200 is positionable through the imaging lumen (discussed below) of the catheter shaft 12.

Turning to FIG. 2A which is a cross-sectional view of the catheter shaft 12 taken along line 2-2 of FIG. 1, the catheter shaft 12, or a portion thereof, may be an extruded shaft having a plurality of lumens formed therein. For example, a proximal portion of the catheter shaft 12 may include the guidewire lumen 24, the inflation lumen 26 (shown as a bifurcated lumen including a first inflation lumen 26a and a second inflation lumen 26b extending parallel to and spaced apart from the first inflation lumen 26a, as well as one or more, or a plurality of auxiliary lumens extending therethrough. For example, the catheter shaft 12 may include an imaging lumen 32 configured to accommodate an intravascular ultrasound (IVUS) device. The guidewire lumen 24 may extend to the distal tip 18 of the catheter shaft 12, while the inflation lumen 26 may extend from a proximal inflation port 34 in the hub assembly 14 into the interior of the expandable balloon 20. For example, the first inflation lumen 26a may be in fluid communication with the first inflatable balloon member 20a and the second inflation lumen 26b may be in fluid communication with the second inflatable balloon member 20b. The first inflation lumen 26a may converge with the second inflation lumen 26a as the inflation lumen 26 extends to the proximal inflation port 34. The imaging lumen 32 extends from a proximal port 36 through the catheter shaft 12 to the expandable balloon 20 and/or distally beyond the expandable balloon 20. In some instances, the imaging lumen 32 may extend to the distal tip 22, while in other instances, the imaging lumen 32 may terminate proximal of the distal tip 18 of the catheter shaft 12. For instance, in some instances the distal termination point of the imaging lumen 32 may be located along the longitudinal length of the expandable balloon 20.

As shown in FIG. 2A, the proximal region of the catheter shaft 12 may have a circular cross-section having an outer diameter D1 of about 0.059 inches. In some instances, the outer diameter D1 may be about 0.06 inches or less, about 0.055 inches or less, or about 0.052 inches or less, for example. In some instances, the outer diameter D1 may be about 0.059 inches, about 0.055 inches, about 0.052 inches, or about 0.050 inches, for example. The outer diameter D1 may be sized such that the catheter shaft 12 of the catheter 10 may be positioned in a 6F guide catheter having an inner lumen diameter of about 0.07 inches, such as in the range of about 0.070 inches to about 0.072 inches, in a 7F guide catheter having an inner lumen diameter of about 0.08 inches, such as in the range of about 0.78 inches to about 0.082 inches, or in an 8F guide catheter having an inner lumen diameter of about 0.09 inches, such as in the range of about 0.088 inches to about 0.091 inches, alongside a trapping balloon catheter, such as the Trapper™ Exchange Device sold by Boston Scientific, Corp., having an outer diameter of about 0.026 inches, for example.

The guidewire lumen 24 may have a diameter D2 of about 0.017 inches to accommodate a 0.014-inch guidewire. The first inflation lumen 26a and the second inflation lumen 26b may each have a diameter D3 of about 0.0115 inches or less, or about 0.0105 inches or less, for example. The imaging lumen 32 may be sized to accommodate an IVUS device, such as the OptiCross™ Imaging Catheter sold by Boston Scientific, Corp., or the IVUS imaging core thereof, while also providing space for fluid aspiration therethrough alongside the IVUS imaging device. The elongate shaft 202 of the imaging device 200 is shown positioned within the imaging lumen 32. For example, in some instances, the IVUS imaging device may have an outer diameter of about 0.025 inches or less. As shown in FIG. 2A, in which the outer diameter D1 is about 0.059 inches, the imaging lumen 32 may have a non-circular cross-section, shown as an elliptical cross-section having a minor radius R1 of about 0.0165 inches and a major radius R2 of about 0.0212 inches, thus providing the imaging lumen 32 with a distance across its minor axis of about 0.0330 inches, a distance across its major axis of about 0.0424 inches, an area of about 0.00110 in², and a perimeter of about 0.1189 inches. In other instances, such as when the outer diameter D1 is about 0.052 inches, the imaging lumen 32 may have an elliptical cross-section having a minor radius R1 of about 0.0130 inches and a major radius R2 of about 0.0175 inches, thus providing the imaging lumen 32 with a distance across its minor axis of about 0.026 inches, a distance across its major axis of about 0.035 inches, an area of about 0.0007147 in², and a perimeter of about 0.09634 inches. The lumens may be sized such that the thickness T of the catheter wall is at least 0.003 inches at all locations of the catheter shaft 12. In other instances, the lumens may be sized such that the thickness T of the catheter wall is at least 0.002 inches, at least 0.0024 inches, at least 0.0026 inches, or at least 0.0028 inches, at all locations of the catheter shaft 12, for example.

As shown in FIG. 2A, the guidewire lumen 24 may have a cross-sectional area larger than the cross-sectional area of each of the first and second inflation lumens 26, and the imaging lumen 32 may have a cross-sectional area larger than the cross-sectional area of the guidewire lumen 24. However, other configurations are contemplated.

In some instances, such as in the embodiment shown in FIG. 2A, the guidewire lumen 24 may be positioned between the first and second inflation lumens 26a, 26b. The guidewire lumen 24 may be equally spaced between the first and second inflation lumens 26a, 26b with a wall thickness of the catheter shaft therebetween of about 0.002 inches or more, or about 0.003 inches or more. As shown in FIG. 2A, the catheter shaft 12 may be constructed with each of the guidewire lumen 24, the first inflation lumen 26a and the second inflation lumen 26b positioned entirely on one side of a plane Y parallel to and passing through the central longitudinal axis X of the catheter shaft 12. The imaging lumen 32 may be positioned such that the central longitudinal axis X of the catheter shaft 12 is within the imaging lumen 32 with a majority of the cross-sectional area of the imaging lumen 32 positioned on the opposite side of the plane Y parallel to and passing through the central longitudinal axis X.

Furthermore, as shown in FIG. 2A, the catheter shaft 12 may be constructed such that a plane Z extending parallel to and passing through the central longitudinal axis X passes through the center of each of the guidewire lumen 24 and the imaging lumen 32. In some instances, the plane Z may be perpendicular to the plane Y. The first inflation lumen 26a may be positioned on a first side of the plane Z and the second inflation lumen 26b may be positioned on a second side of the plane Z. In some instances, the first and second inflation lumens 26a, 26b may be equidistantly spaced from plane Z.

FIG. 2B illustrates an alternative cross-sectional view of a proximal portion of the catheter shaft 12 taken along line 2-2 of FIG. 1. The outer diameter D1, as well as the size, shape, configuration and position of the guidewire lumen 24 and the first and second inflation lumens 26a, 26b may be the same as described above regarding FIG. 2A. The imaging lumen 32 may be sized to accommodate an IVUS device, such as the OptiCross™ Imaging Catheter sold by Boston Scientific, Corp., or the IVUS imaging core thereof, while also providing space for fluid aspiration therethrough alongside the IVUS imaging device. The elongate shaft 202 of the imaging device 200 is shown positioned within the imaging lumen 32. For example, in some instances, the IVUS imaging device may have an outer diameter of about 0.025 inches or less. As shown in FIG. 2B, in which the outer diameter D1 is about 0.059 inches, the imaging lumen 32 may have a non-circular cross-section, shown as an oblong cross-section having a minor radius R1 of about 0.0165 inches and a major radius R2 of greater than 0.0212 inches, thus providing the imaging lumen 32 with a distance across its minor axis of about 0.0330 inches, a distance across its major axis of greater than 0.043 inches, an area of about 0.00119 in², and a perimeter of about 0.1270 inches. In other instances, such as when the outer diameter D1 is about 0.052 inches, the imaging lumen 32 may have an oblong cross-section having a minor radius R1 of about 0.0130 inches and a major radius R2 of greater than 0.0175 inches, thus providing the imaging lumen 32 with a distance across its minor axis of about 0.026 inches, a distance across its major axis of greater than 0.035 inches, an area of about 0.00081 in², and a perimeter of about 0.1064 inches. The lumens may be sized such that the thickness T of the catheter wall is at least 0.003 inches at all locations of the catheter shaft 12. In other instances, the lumens may be sized such that the thickness T of the catheter wall is at least 0.002 inches, at least 0.0024 inches, at least 0.0026 inches, or at least 0.0028 inches, at all locations of the catheter shaft 12, for example.

As shown in FIG. 2B, the guidewire lumen 24 may have a cross-sectional area larger than the cross-sectional area of each of the first and second inflation lumens 26, and the imaging lumen 32 may have a cross-sectional area larger than the cross-sectional area of the guidewire lumen 24. However, other configurations are contemplated.

As shown in FIG. 2B, the catheter shaft 12 may be constructed with each of the guidewire lumen 24, the first inflation lumen 26a and the second inflation lumen 26b positioned entirely on one side of a plane Y parallel to and passing through the central longitudinal axis X of the catheter shaft 12. The imaging lumen 32 may be positioned such that the central longitudinal axis X of the catheter shaft 12 is within the imaging lumen 32 with a majority of the cross-sectional area of the imaging lumen 32 positioned on the opposite side of the plane Y parallel to and passing through the central longitudinal axis X.

Furthermore, as shown in FIG. 2B, the catheter shaft 12 may be constructed such that a plane Z extending parallel to and passing through the central longitudinal axis X passes through the center of each of the guidewire lumen 24 and the imaging lumen 32. In some instances, the plane Z may be perpendicular to the plane Y. The first inflation lumen 26a may be positioned on a first side of the plane Z and the second inflation lumen 26b may be positioned on a second side of the plane Z. In some instances, the first and second inflation lumens 26a, 26b may be equidistantly spaced from plane Z.

FIG. 2C illustrates an alternative cross-sectional view of a proximal portion of the catheter shaft 12 taken along line 2-2 of FIG. 1. The proximal region of the catheter shaft 12 may having a circular cross-section having an outer diameter D1 of about 0.052 inches, for example. In some instances, the outer diameter D1 may be about 0.06 inches or less, about 0.055 inches or less, or about 0.052 inches or less, for example. In some instances, the outer diameter D1 may be about 0.059 inches, about 0.055 inches, about 0.052 inches, or about 0.050 inches, for example. The outer diameter D1 may be sized such that the catheter shaft 12 of the catheter 10 may be positioned in a 6F guide catheter having an inner lumen diameter of about 0.070 inches, in a 7F guide catheter having an inner lumen diameter of about 0.078 inches, or in an 8F guide catheter having an inner lumen diameter of about 0.088 inches, alongside a trapping balloon catheter, such as the Trapper™ Exchange Device sold by Boston Scientific, Corp., having an outer diameter of about 0.026 inches, for example.

The guidewire lumen 24 may have a diameter D2 of about 0.017 inches to accommodate a 0.014-inch guidewire. The catheter shaft 12 may have a single inflation lumen 26 having a diameter D3 of about 0.013 inches or less, for example. The imaging lumen 32 may be sized to accommodate an IVUS device, such as the OptiCross™ Imaging Catheter sold by Boston Scientific, Corp., or the IVUS imaging core thereof, while also providing space for fluid aspiration therethrough alongside the IVUS imaging device. The elongate shaft 202 of the imaging device 200 is shown positioned within the imaging lumen 32. For example, in some instances, the IVUS imaging device may have an outer diameter of about 0.025 inches or less. As shown in FIG. 2C, in which the outer diameter D1 is about 0.052 inches, the imaging lumen 32 may have an elliptical cross-section having a minor radius R1 of about 0.0125 inches and a major radius R2 of about 0.017 inches, thus providing the imaging lumen 32 with a distance across its minor axis of about 0.025 inches, a distance across its major axis of about 0.034 inches, an area of about 0.0006676 in², and a perimeter of about 0.09322 inches. In other instances, such as when the outer diameter D1 is about 0.059 inches, the imaging lumen 32 may have a non-circular cross-section, shown as an elliptical cross-section having a minor radius R1 of about 0.0165 inches and a major radius R2 of about 0.0212 inches, thus providing the imaging lumen 32 with a distance across its minor axis of about 0.0330 inches, a distance across its major axis of about 0.0424 inches, an area of about 0.00110 in², and a perimeter of about 0.1189 inches The lumens may be sized such that the thickness T of the catheter wall is at least 0.003 inches at all locations of the catheter shaft 12. In other instances, the lumens may be sized such that the thickness T of the catheter wall is at least 0.002 inches, at least 0.0024 inches, at least 0.0026 inches, or at least 0.0028 inches, at all locations of the catheter shaft 12, for example.

As shown in FIG. 2C, the guidewire lumen 24 may have a cross-sectional area larger than the cross-sectional area of the inflation lumen 26, and the imaging lumen 32 may have a cross-sectional area larger than the cross-sectional area of the guidewire lumen 24. However, other configurations are contemplated.

As shown in FIG. 2C, the catheter shaft 12 may be constructed with each of the guidewire lumen 24 and the inflation lumen 26 positioned entirely on one side of a plane Y parallel to and passing through the central longitudinal axis X of the catheter shaft 12. The imaging lumen 32 may be positioned such that the central longitudinal axis X of the catheter shaft 12 is within the imaging lumen 32 with a majority of the cross-sectional area of the imaging lumen 32 positioned on the opposite side of the plane Y parallel to and passing through the central longitudinal axis X.

FIG. 2D illustrates an alternative cross-sectional view of a proximal portion of the catheter shaft 12 taken along line 2-2 of FIG. 1. The proximal region of the catheter shaft 12 may having a circular cross-section having an outer diameter D1 of about 0.059 inches, for example. In some instances, the outer diameter D1 may be about 0.06 inches or less, about 0.055 inches or less, or about 0.052 inches or less, for example. In some instances, the outer diameter D1 may be about 0.059 inches, about 0.055 inches, about 0.052 inches, or about 0.050 inches, for example. The outer diameter D1 may be sized such that the catheter shaft 12 of the catheter 10 may be positioned in a 6F guide catheter having an inner lumen diameter of about 0.070 inches, in a 7F guide catheter having an inner lumen diameter of about 0.078 inches, or in an 8F guide catheter having an inner lumen diameter of about 0.088 inches, alongside a trapping balloon catheter, such as the Trapper™ Exchange Device sold by Boston Scientific, Corp., having an outer diameter of about 0.026 inches, for example.

The guidewire lumen 24 may have a diameter D2 of about 0.017 inches to accommodate a 0.014-inch guidewire. The first inflation lumen 26a and the second inflation lumen 26b may each have a diameter D3 of about 0.0115 inches or less, or about 0.0105 inches or less, for example. The imaging lumen 32 may be sized to accommodate an IVUS device, such as the OptiCross™ Imaging Catheter sold by Boston Scientific, Corp., or the IVUS imaging core thereof. The elongate shaft 202 of the imaging device 200 is shown positioned within the imaging lumen 32. For example, in some instances, the IVUS imaging device may have an outer diameter of about 0.025 inches or less. As shown in FIG. 2D, in which the outer diameter D1 is about 0.059 inches, the imaging lumen 32 may have a circular cross-section having a radius of about 0.0125 inches (diameter of about 0.025 inches). Th catheter shaft 12 may include an additional lumen, such as an aspiration lumen 38 extending parallel to and spaced apart from the imaging lumen 32. The aspiration lumen 38 may have a circular cross-section in some embodiments. In embodiments in which the outer dimeter D1 is about 0.059 inches, the aspiration lumen 38 may have a radius of about 0.01125 inches (diameter of about 0.0225 inches). The lumens may be sized such that the thickness T of the catheter wall is at least 0.003 inches at all locations of the catheter shaft 12. In other instances, the lumens may be sized such that the thickness T of the catheter wall is at least 0.002 inches, at least 0.0024 inches, at least 0.0026 inches, or at least 0.0028 inches, at all locations of the catheter shaft 12, for example.

As shown in FIG. 2D, the guidewire lumen 24 may have a cross-sectional area larger than the cross-sectional area of each of the first and second inflation lumens 26, and the imaging lumen 32 may have a cross-sectional area larger than the cross-sectional area of the guidewire lumen 24. The aspiration lumen 38 may have a cross-sectional lumen greater than or less than the cross-sectional area of the guidewire lumen 24 and/or the imaging lumen 32. However, other configurations are contemplated.

As shown in FIG. 2D, the catheter shaft 12 may be constructed with each of the guidewire lumen 24 and the first and second inflation lumens 26a, 26b positioned entirely on one side of a plane Y parallel to and passing through the central longitudinal axis X of the catheter shaft 12. The imaging lumen 32 and the aspiration lumen 38 may be positioned such that the plane Y passes through each of the imaging lumen 32 and the aspiration lumen 38 with a majority of the cross-sectional area of the imaging lumen 32 and a majority of the cross-sectional area of the aspiration lumen 38 positioned on the opposite side of the plane Y parallel to and passing through the central longitudinal axis X.

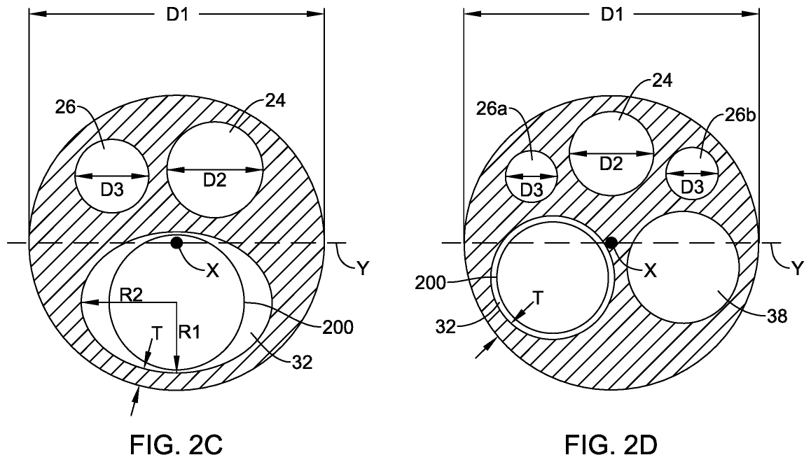

Turning to FIG. 3 which is a cross-sectional view of the catheter shaft 12 taken along line 3-3 of FIG. 1, the catheter shaft 12, or a portion thereof, may be an extruded shaft having a plurality of lumens formed therein. For example, a distal portion of the catheter shaft 12 may include the guidewire lumen 24, the inflation lumen 26 (shown as a bifurcated lumen including a first inflation lumen 26a and a second inflation lumen 26b extending parallel to and spaced apart from the first inflation lumen 26b, as well as one or more, or a plurality of auxiliary lumens extending therethrough. For example, the distal portion of the catheter shaft 12 may include the imaging lumen 32 configured to accommodate an intravascular ultrasound (IVUS) imaging device and/or an aspiration lumen (See FIG. 2D). The elongate shaft 202 of the imaging device 200 is shown positioned within the imaging lumen 32.

The cross-sectional shape of the catheter shaft 12 in the distal portion of the catheter shaft 12 may be different than the cross-sectional shape of the catheter shaft 12 in the proximal portion of the catheter shaft 12. For example, as shown above with regard to FIGS. 2A-2D, the proximal portion of the catheter shaft 12 may have a circular cross-sectional shape, whereas the distal portion of the catheter shaft 12 may have a non-circular cross-sectional shape, such as a generally rectangular cross-sectional shape.

The position and arrangement of the various lumens in the proximal portion of the catheter shaft 12 may be dissimilar from the position and arrangement of the lumens in the distal portion of the catheter shaft 12. For example, in the distal portion of the catheter shaft 12 the first and second inflation lumens 26a, 26b may be positioned on opposite sides of the imaging lumen 32 and the guidewire lumen 24, with the imaging lumen 32 and the guidewire lumen 24 positioned between the first and second inflation lumens 26a, 26b. The imaging lumen 32, the guidewire lumen 24, and the first and second inflation lumens 26a, 26b may be positioned (e.g., aligned) such that the plane Y that extends parallel to and passes through the central longitudinal axis X of the catheter shaft 12 passes through each of the imaging lumen 32, the guidewire lumen 24, and the first and second inflation lumens 26a, 26b. In some instances, the plane Y may pass through the center, or near the center, of each of the imaging lumen 32 and the guidewire lumen 24.

The overall width W, measured parallel to the plane Y, of the distal portion of the catheter shaft 12 may be about 0.07 inches or less, about 0.068 inches or less, 0.066 inches or less, or 0.065 inches or less, for example. The overall height H, measured perpendicular to the width W, of the distal portion of the catheter shaft 12 may be about 0.035 inches or less, about 0.033 inches or less, or about 0.032 inches or less, for example.

The guidewire lumen 24 may have a diameter D2 of about 0.017 inches to accommodate a 0.014-inch guidewire. The imaging lumen 32 may have a diameter D4 of about 0.026 inches to accommodate an IVUS imaging catheter. Each of the first and second inflation lumens 26a, 26b may have a width D5 of about 0.0037 inches. The lumens may be sized such that the thickness T of the catheter wall is at least 0.003 inches at all locations of the catheter shaft 12. In other instances, the lumens may be sized such that the thickness T of the catheter wall is at least 0.002 inches, at least 0.0024 inches, at least 0.0026 inches, or at least 0.0028 inches, at all locations of the catheter shaft 12, for example.

The extruded proximal portion of the catheter shaft 12 may be joined to the extruded distal portion of the catheter shaft 12 such that the lumens of the proximal portion of the catheter shaft 12 are in communication with the corresponding lumens of the distal portion of the catheter shaft 12. In other words, the lumens described above, may be continuous through the proximal and distal portions of the catheter shaft 12 while the cross-sectional shape of the proximal portion of the catheter shaft 12 may be different from the cross-sectional shape of the distal portion of the catheter shaft 12.

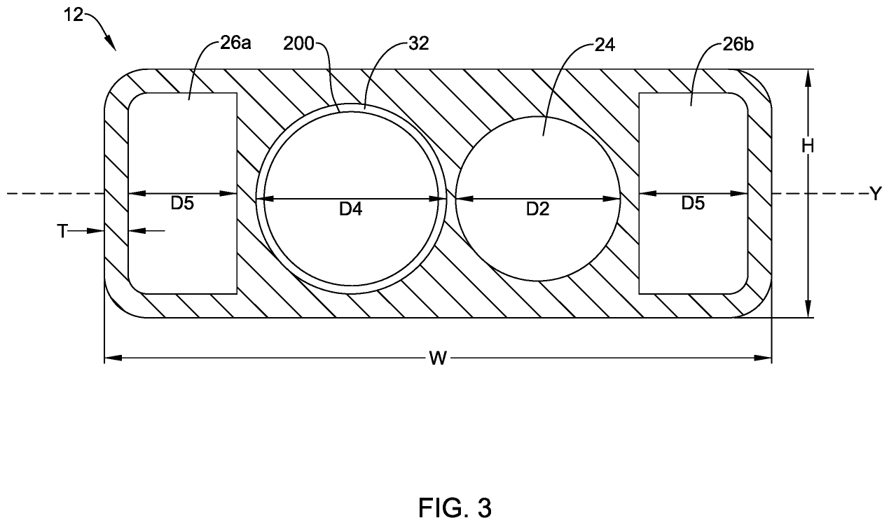

Turning to FIG. 4 which is a cross-sectional view of the distal end region of the catheter shaft 12 taken along line 4-4 of FIG. 1, including the expandable balloon 20 of the catheter shaft 12. In some instances, the first and second inflation balloon members 20a, 20b of the expandable balloon 20 may be formed as a unitary or monolithic portion of the catheter shaft 12. However, in other instances, the first and second inflation balloon members 20a, 20b of the expandable balloon 20 may be formed separately and then secured to the catheter shaft 12. As shown in FIG. 4, the first inflation lumen 26a may open into the interior of the first inflatable balloon member 20a and the second inflation lumen 26b may open into the interior of the second inflatable balloon member 20b.

The overall width W1, measured parallel to the plane Y, of the expandable balloon 20, including both the first and second inflatable balloon members 20a, 20b, when inflated may be about 0.142 inches. In some instances, the overall width W1 may be about 0.14 inches or more, about 0.16 inches or more, about 0.18 inches or more, about 0.20 inches or more, about 0.14 inches, about 0.16 inches, about 0.18 inches, about 0.20 inches, in the range of about 0.14 inches to about 0.20 inches, or in the range of about 0.16 inches to about 0.22 inches, for example. The overall height H1, measured perpendicular to the width W1, of the expandable balloon 20, (e.g., the height of either the first and/or second inflatable balloon members 20a, 20b), when inflated may be about 0.023 inches, for example. Thus, the width of the inflatable balloon 20 may be greater than the height of the inflatable balloon 20 when inflated. For instance, when inflated the expandable balloon 20 may have a width to height ratio of 2:1 or more, 3:1 or more, 4:1 or more, 5:1 or more, or 6:1 or more.

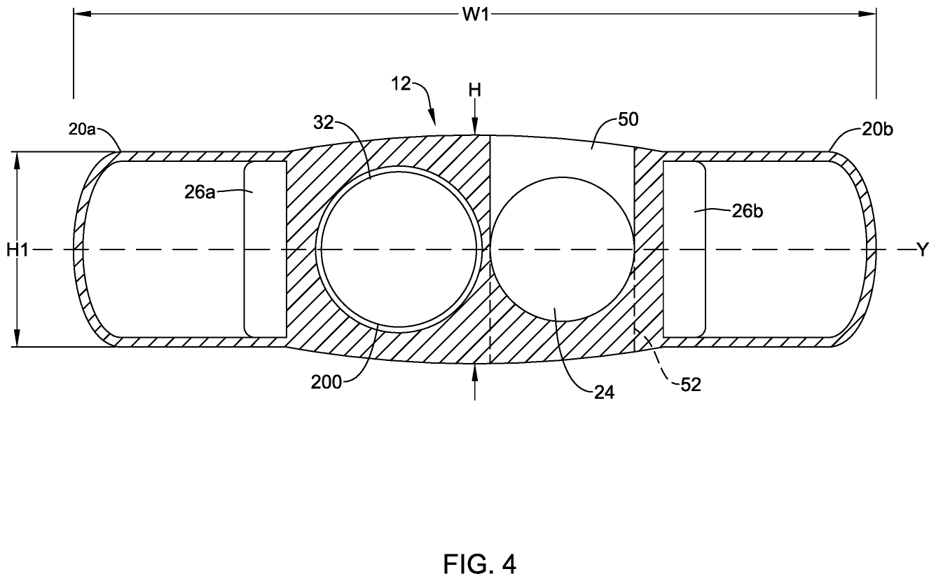

The distal region of the elongate shaft 12 may also include a first lateral opening 50 opening to an exterior of the catheter shaft 12 between the first inflatable balloon member 26a and the second inflatable balloon member 26b. In some instances, the distal region of the elongate shaft 12 may also include a second lateral opening 52 (see also FIG. 5) opening to an exterior of the catheter shaft 12 between the first inflatable balloon member 26a and the second inflatable balloon member 26b. Each of the first lateral opening 50 and the second lateral opening 52 may be in communication with the guidewire lumen 24. The first lateral opening 50 and the second lateral opening 52 may each extend through a sidewall of the catheter shaft 12. For instance, the first lateral opening 50 may extend through a sidewall of the catheter shaft 12 on a first side of the catheter shaft 12 exposed between the first and second inflatable balloon members 26a, 26b, and the second lateral opening 52 may extend through a sidewall of the catheter shaft 12 on an opposite, second side of the catheter shaft 12 exposed between the first and second inflatable balloon members 26a, 26b.

Each of the first lateral opening 50 and the second lateral opening 52 may be configured to permit a re-entry device or penetration member to be advanced therefrom during a recanalization procedure to puncture through a tissue layer of a vessel wall from the subintimal space into the true lumen of a blood vessel. For example, a distal portion of a re-entry device or penetration member, which may be advanced through the guidewire lumen 24, may pass laterally from the catheter shaft 12 out through either the first lateral opening 50 or the second lateral opening 52 to penetrate through a tissue layer of a blood vessel.

As shown in FIG. 5, in some instances, the first lateral opening 50 may be located in the distal region of the catheter shaft 12 at a location between the proximal end and the distal end of the expandable balloon 20, e.g., between proximal ends and the distal ends of the first and second inflatable balloon members 20a, 20b. Additionally, the second lateral opening 52 may be located in the distal region of the catheter shaft 12 at a location between the proximal end and the distal end of the expandable balloon 20, e.g., between proximal ends and the distal ends of the first and second inflatable balloon members 20a, 20b. In other instances, the first lateral opening 50 and/or the second lateral opening 52 may be otherwise located in the distal end region of the catheter shaft 12, such as proximal of the proximal end of the expandable balloon 20 or distal of the distal end of the expandable balloon 20. The first lateral opening 50 and the second lateral opening 52 may be longitudinally offset (i.e., axially spaced apart) from one another along the catheter shaft 12. For instance, the first lateral opening 50 may be located proximal of the second lateral opening 52. The catheter shaft 12 may also include a radiopaque marker 51 located proximate the first lateral opening 50 to provide an indication of the location of the first lateral opening 50 under fluoroscopy. Additionally, the catheter shaft 12 may include a radiopaque marker 53 located proximate the second lateral opening 52 to provide an indication of the location of the second lateral opening 52 under fluoroscopy.

Also shown in FIG. 5 is a guidewire 100 positioned in the guidewire lumen 24 of the catheter shaft 12 and an IVUS imaging device 200 (e.g., an IVUS imaging catheter or core) including an ultrasound transducer 210 at a distal end thereof positioned in the imaging lumen 32 of the catheter shaft 12. The IVUS imaging device 200 may be longitudinally moveable within the imaging lumen 32 such that the imaging device 200 may be advanced distally relative to the catheter shaft 12 and withdrawn proximally relative to the catheter shaft 12 to position the ultrasound transducer 210 at a desired location within the imaging lumen 32 during a medical procedure. In other instances, the IVUS imaging device 200 may be immovably fixed longitudinally within the imaging lumen 32. In some embodiments, the ultrasound transducer 210 may be positioned or positionable distal of the first lateral opening 50, distal of the second lateral opening 52 and/or distal of the expandable balloon 20 such that the imaging device can provide an ultrasound image of the trajectory of a re-entry device from the catheter shaft 12.

In some instances, it may be undesired, difficult or impossible to pass through an occlusion, such as a chronic total occlusion (CTO) in a lumen of a blood vessel with a medical device to recanalize the vessel. In such instances, it may be possible to recanalize the blood vessel through a subintimal approach using the catheter 10. As will be described further herein, the catheter 10 may be configured to facilitate re-entry into a true lumen of a blood vessel distal of an occlusion (e.g., chronic total occlusion) in the blood vessel from a subintimal spaced formed in the vessel wall bypassing the occlusion. For instance, the distal portion of the catheter shaft 12, including the expandable balloon 20, may be advanced into a subintimal space (i.e., a space between the intima layer and the adventitia layer) created in the vessel wall of a blood vessel, such as through dissection of the tissue layers of the vessel wall. Once positioned in the subintimal space, the first and second inflatable balloon members 20a, 20b of the expandable balloon 20 may be inflated to an expanded/inflated configuration between the intima layer and the adventitia layer of the vessel wall. As the first and second balloon members 20a, 20b are inflated to the expanded/inflated configuration, first and second balloon members 20a, 20b extend laterally from the catheter shaft 12 within the subintimal space formed in the vessel wall to automatically orient the either the first lateral opening 50 or the second lateral opening 52 radially inward toward the true lumen of the blood vessel. A re-entry device (e.g. a re-entry guidewire or other penetration member) may be advanced through the guidewire lumen 24 and out through one of the first lateral opening 50 or second lateral opening 52 (whichever one is facing the true lumen) to penetrate through the intima layer into the true lumen from the subintimal location.

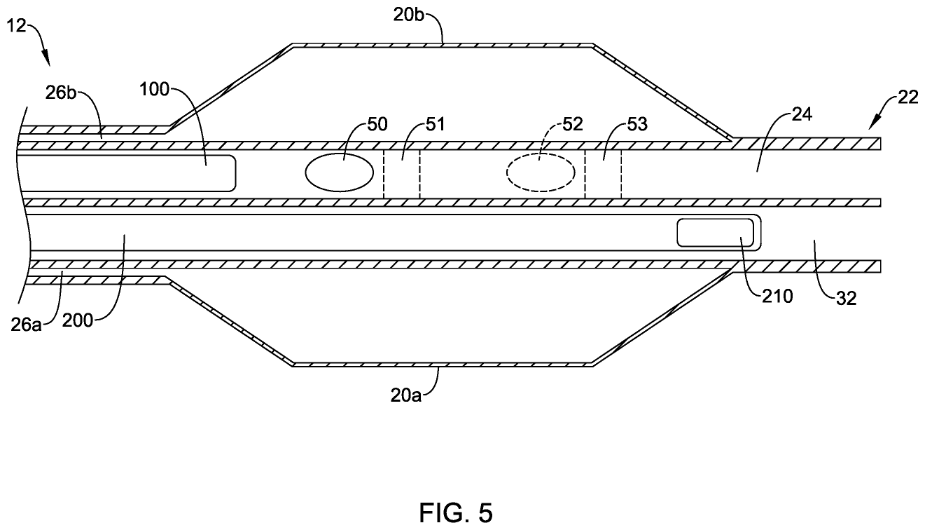

Turning to FIGS. 6-10, several aspects of an exemplary method for recanalizing an occluded blood vessel 80 using the catheter 10 are illustrated. The blood vessel 80 typically has three tissue layers, an innermost layer or intima layer (i.e., tunica intima) 82, an intermediate layer or media layer (i.e., tunica media) 84, and an outermost layer or adventitia layer (tunica adventitia) 86, with the media layer 84 positioned between the intima layer 82 and the adventitia layer 86. The intima layer 82 is a layer of endothelial cells lining the lumen 88 of the vessel 80, as well as a subendothelial layer made up of mostly loose connective tissue. The media layer 84 is a muscular layer formed primarily of circumferentially arranged smooth muscle cells. The adventitia layer 86, which forms the exterior layer of the vessel wall 80 is formed primarily of loose connective tissue made up of fibroblasts and associated collagen fibers.

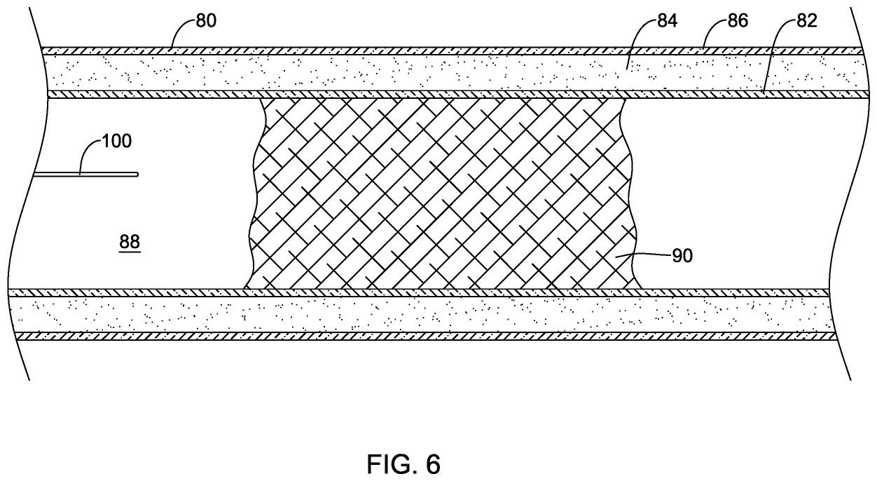

As shown in FIG. 6, a guidewire 100 may initially be advanced through the lumen 88 of the vessel 80 to a location proximate a proximal end of an occlusion 90 blocking the lumen 88. The guidewire 100 may then be advanced to penetrate outward through the intima layer 82 at a location proximal of the proximal end of the occlusion 90 into the wall of the vessel 80. With the tip of the guidewire 100 located between the intima layer 82 and the adventitia layer 86, the guidewire 100 may be further advanced distally in a subintimal manner to create a subintimal space between the intima layer 82 and the adventitia layer 86. As shown in FIG. 7, the guidewire 100 may be advanced in a subintimal manner until the distal tip of the guidewire 100 is located distal of the distal end of the occlusion 90 in the subintimal space created, such as by dissection of the tissue layers of the wall of the vessel 80. In some instances, another catheter device may be initially used to exit the lumen 88 proximal of the occlusion 90 and form a subintimal space. In such an instance, the guidewire 100 may be subsequently advanced through the catheter into the subintimal space and the catheter may be withdrawn, leaving the guidewire 100 positioned in the subintimal space as shown in FIG. 7.

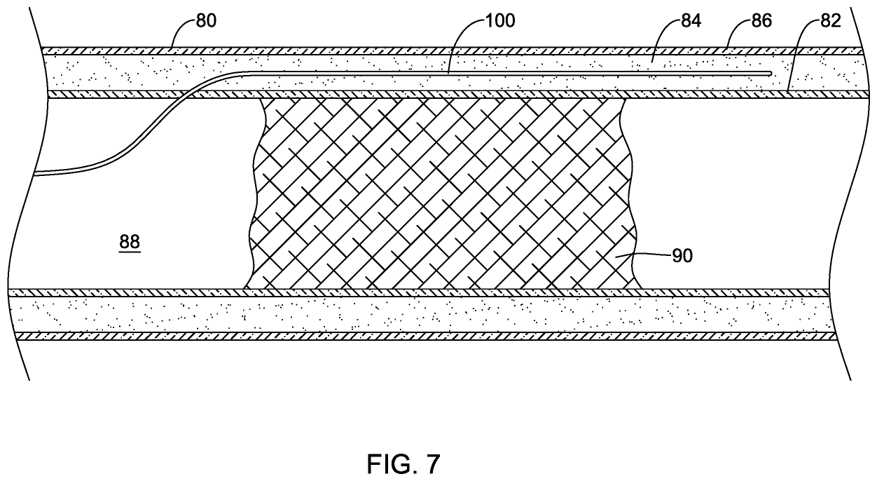

The recanalization catheter 10 may then be advanced distally over the guidewire 100 from the true lumen 88 proximal of the occlusion 90, into the subintimal space between the intima layer 82 and the adventitia layer 86, to a position in the subintimal space in which the distal portion of the catheter 10, including the expandable balloon 20, is located distal of the distal end of the occlusion 90, as shown in FIG. 8. The recanalization catheter 10 may be advanced through the subintimal space in a delivery configuration, such as with the expandable balloon 20 in a deflated and folded configuration.

The IVUS imaging device 200 may be advanced through the imaging lumen 32, or otherwise positioned within the imaging lumen 32, to position the imaging transducer 210 distal of the occlusion 90 to obtain ultrasound images of the true lumen 88 and confirm that the recanalization catheter 10 has been advanced sufficiently past the occlusion 90 for successful re-entry into the true lumen 88 with a re-entry device advanced from the recanalization catheter 10. The ultrasound transducer 210 may be positionable distal of the first lateral opening 50, the second lateral opening 52 and/or distal of the expandable balloon 20 such that the imaging device can provide an ultrasound image of the trajectory of a re-entry device from the catheter shaft 12. In some instances, the imaging device 200 may be advanced with the catheter 10 into the subintimal space. In other instances, the imaging device 200 may be advanced through the imaging lumen 32 subsequent to advancing the recanalization catheter 10 into the subintimal space.

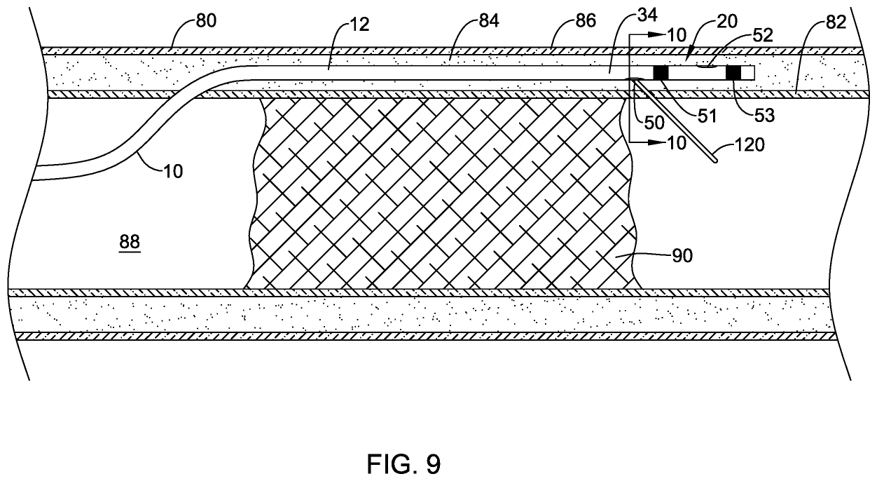

Additionally, once positioned in the subintimal space, the first and second inflatable balloon members 20a, 20b of the expandable balloon 20 may be inflated to an expanded/inflated configuration between the intima layer and the adventitia layer of the vessel wall, as shown in FIG. 10. The first and second inflatable balloon members 20a, 20b may be inflated prior to or subsequent to using the imaging device 200 to confirm desired placement of the recanalization catheter 10 distally past the occlusion 90. As the first and second balloon members 20a, 20b are inflated to the expanded/inflated configuration, first and second balloon members 20a, 20b extend laterally from the catheter shaft 12 within the subintimal space formed in the vessel wall to automatically orient either the first lateral opening 50 or the second lateral opening 52 radially inward toward the true lumen 88 of the blood vessel 80. As shown in FIG. 10, expansion/inflation of the inflatable balloon members 20a, 20b may rotationally orient the catheter shaft 12 in one of two possible orientations, either with the first lateral opening 50 facing the true lumen 88 of the blood vessel 80 or with the second lateral opening 52 facing the true lumen 88 of the blood vessel 80.

If desired, blood within the subintimal space may be aspirated through the imaging lumen exterior of the IVUS imaging device 200 (or through the separate aspiration lumen 38 if provided therewith) to reduce the pressure in the subintimal space and/or draw the intima layer 86 against the catheter shaft 12. Aspiration of blood from the subintimal space may also prevent the true lumen 88 distal of the occlusion 90 from collapsing.

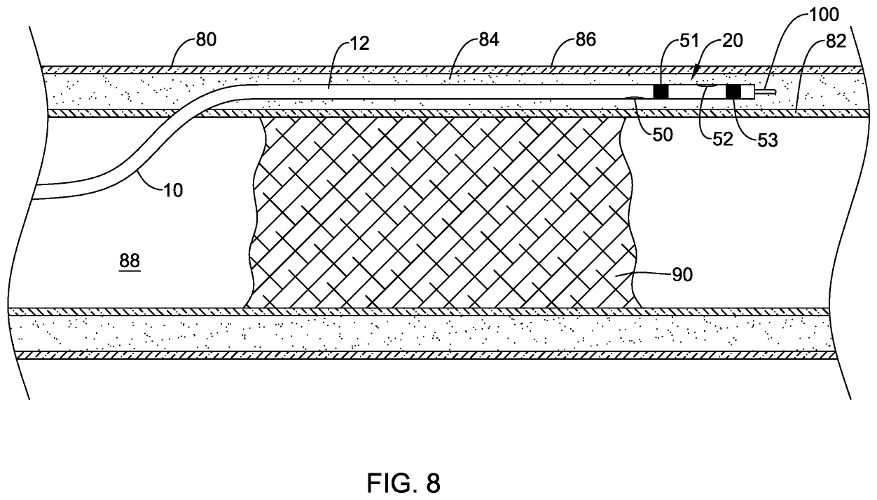

The guidewire 100 may be withdrawn from the guidewire lumen 24, and an elongate re-entry device or penetration member 120 may then be advanced through the guidewire lumen 24 of the catheter 10 and exit the first lateral opening 50, if the first lateral opening 50 is facing the true lumen 88, to penetrate through the intima layer 82 into the true lumen 88 distal of the occlusion 90, shown in FIG. 9. It is noted that if the expandable balloon 20 was inflated such that the catheter shaft 12 was oriented with the second lateral opening 52 facing the true lumen 88, then the elongate re-entry device or penetration member 120 may be advanced through the guidewire lumen 24 of the catheter 10 and exit the second lateral opening 52 to penetrate through the intima layer 82 into the true lumen 88 distal of the occlusion 90. In some embodiments, the re-entry device or penetration member 120 may be the guidewire 100, or another guidewire introduced through the guidewire lumen 24 of the catheter shaft 12. In some instances, the re-entry device may have a pre-formed bent distal end portion in which the distal end portion is bent at an oblique angle to a proximal portion of the re-entry device. The bent distal end portion may facilitate exiting the first lateral opening 50 or second lateral opening 52 when the distal tip of the re-entry device or penetration member 120 encounters the lateral opening. In other instances, the recanalization catheter 10 may include a deflection mechanism to deflect the re-entry device or penetration member 120 out through the first lateral opening 50 or second lateral opening 52. In some instances, the re-entry device or penetration member 120 may include a reduced diameter penetrating tip 125 (shown in FIG. 10) to facilitate penetrating through the intima layer 82. In other embodiments, the re-entry device or penetration member 120 may be an elongate member, such as a needle cannula or stylet, having a sharpened distal tip configured to pierce through the intima layer 82 into the lumen 88 distal of the occlusion 90.

In some instances, fluoroscopy may be utilized to confirm the trajectory of the re-entry or penetration member 120 from the catheter shaft 12 to ensure the re-entry or penetration member 120 is being advanced toward the true lumen 88 and not radially outward through the adventitia layer 86. Additionally or alternatively, the IVUS imaging device 200 may be utilized to obtain ultrasound images of the true lumen 88 and confirm the trajectory of the re-entry or penetration member 120 to ensure the re-entry or penetration member 120 is being advanced toward the true lumen 88. Due to the placement of the imaging lumen 32 laterally beside the guidewire lumen 24 such that the IVUS imaging device 200 is positioned laterally adjacent the re-entry or penetration member 120, the presence of the re-entry or penetration member 120 does not obstruct or interfere with the imaging capabilities of the IVUS transducer 210 such that the IVUS transducer 210 is able to obtain a clear image through an angle of about 90 to about 150 degrees toward the true lumen 88.

In the event the re-entry device or penetration member 120 is a guidewire, the catheter 10 may be withdrawn while leaving the guidewire routed around the occlusion 90 via the subintimal pathway. In instances in which the re-entry device or penetration member 120 is a separate elongate member, such as a needle cannula or stylet, the re-entry device or penetration member 120 may be withdrawn and replaced with a guidewire. The catheter 10 and/or penetration member 120 may be withdrawn while leaving the guidewire routed around the occlusion 90 via the subintimal pathway.

In some instances, a trapping balloon catheter may be used to maintain the position of the re-entry device/penetration member 120 (or subsequently placed guidewire) through the subintimal pathway around the occlusion 90 while the recanalization catheter 10 is withdrawn. For instance, the recanalization catheter 10 may have been advanced through a guide catheter, such as a 6F, 7F or 8F guide catheter, to reach the subintimal space alongside the occlusion 90 as shown in FIG. 8. Prior to withdrawing the recanalization catheter 10 from the vessel subsequent to successful re-entry into the true lumen distal of the occlusion 90, a trapping balloon catheter may be advanced through the guide catheter alongside an exterior of the recanalization catheter 10 and then the trapping balloon of the trapping balloon catheter may be inflated distal of the recanalization catheter 10 within the guide catheter to secure the re-entry device/penetration member 120 or other guidewire against the inner wall of the guide catheter. With the re-entry device/penetration member 120 or other guidewire firmly secured to the inner wall of the guide catheter to prevent longitudinal movement thereof, the recanalization catheter 10 may be withdrawn from the guide catheter and the vessel 80. Once the recanalization catheter 10 is withdrawn, the trapping balloon of the trapping balloon catheter may be deflated, leaving the re-entry device/penetration member 120 or other guidewire in place.

Once a pathway has been created across the occlusion 90, (e.g., around the occlusion 90 via a subintimal track), one or more additional medical devices may be advanced through the blood vessel 80 over the re-entry device/penetration member 120 or other guidewire to enlarge the pathway and/or pass distally of the occlusion 90 to perform a further medical procedure.

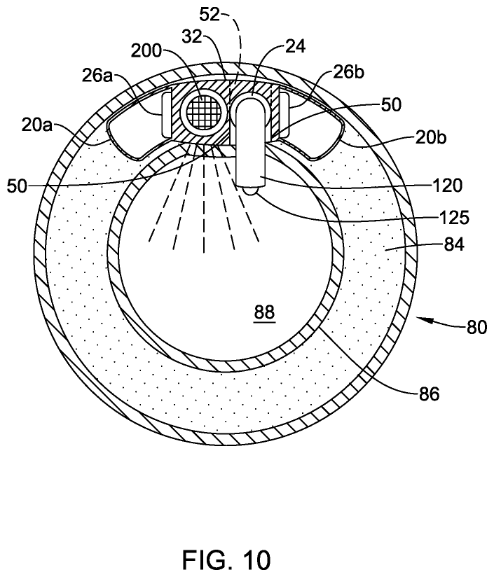

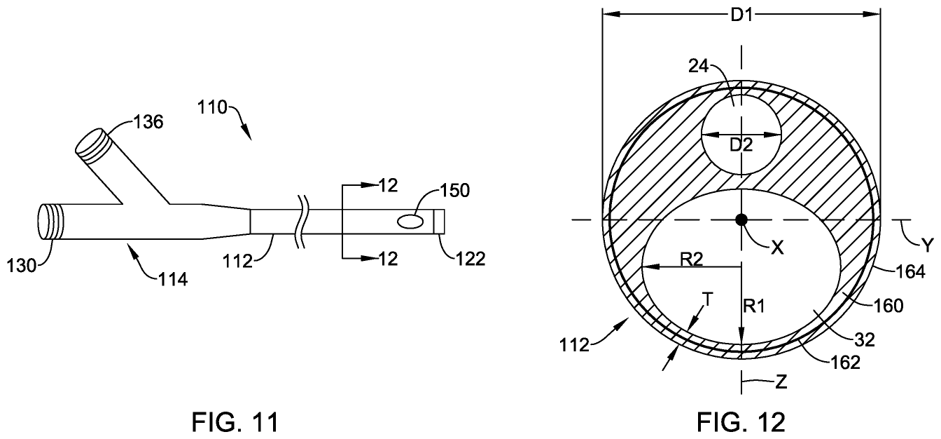

FIG. 11 is a plan view of an exemplary catheter 110 for recanalization of a blood vessel. The catheter 110 may include an elongate catheter shaft 112 extending distally from a hub assembly 114 to a distal tip 122 of the catheter shaft 112. As shown in the cross-sectional view of FIG. 12, the catheter shaft 112 may include a guidewire lumen 24 and an imaging lumen 32 extending therein. The distal end region of the catheter shaft 112 may include a lateral opening 150 in communication with the guidewire lumen 24. The guidewire lumen 24 may extend from the proximal guidewire port 130 to the distal tip 122 of the catheter shaft 12, in some instances, or the guidewire lumen 24 may terminate at the lateral opening 150. The imaging lumen 32 may extend from a proximal port 36 through the catheter shaft 12 distally beyond the lateral opening 150. In some instances, the imaging lumen 32 may extend to the distal tip 122 or the imaging lumen 32 may terminate proximal of the distal tip 122.

As shown in FIG. 12, the catheter shaft 112 may have a circular cross-section having an outer diameter D1 of about 0.059 inches. In some instances, the outer diameter D1 may be about 0.06 inches or less, about 0.055 inches or less, or about 0.052 inches or less, for example. In some instances, the outer diameter D1 may be about 0.059 inches, about 0.055 inches, about 0.052 inches, or about 0.050 inches, for example. The outer diameter D1 may be sized such that the catheter shaft 112 of the catheter 110 may be positioned in a 6F guide catheter having an inner lumen diameter of about 0.07 inches, such as in the range of about 0.070 inches to about 0.072 inches, in a 7F guide catheter having an inner lumen diameter of about 0.08 inches, such as in the range of about 0.78 inches to about 0.082 inches, or in an 8F guide catheter having an inner lumen diameter of about 0.09 inches, such as in the range of about 0.088 inches to about 0.091 inches, alongside a trapping balloon catheter, such as the Trapper™ Exchange Device sold by Boston Scientific, Corp., having an outer diameter of about 0.026 inches, for example.

The guidewire lumen 24 may have a diameter D2 of about 0.017 inches to accommodate a 0.014-inch guidewire. The imaging lumen 32 may be sized to accommodate an IVUS device, such as the OptiCross™ Imaging Catheter sold by Boston Scientific, Corp., or the IVUS imaging core thereof, while also providing space for fluid aspiration therethrough alongside the IVUS imaging device. For example, in some instances, the IVUS imaging device may have an outer diameter of about 0.025 inches or less.

As shown in FIG. 12, in which the outer diameter D1 is about 0.059 inches, the imaging lumen 32 may have a non-circular cross-section, shown as an elliptical cross-section having a minor radius R1 of about 0.0165 inches and a major radius R2 of about 0.0212 inches, thus providing the imaging lumen 32 with a distance across its minor axis of about 0.0330 inches, a distance across its major axis of about 0.0424 inches, an area of about 0.00110 in², and a perimeter of about 0.1189 inches. In other instances, such as when the outer diameter D1 is about 0.052 inches, the imaging lumen 32 may have an elliptical cross-section having a minor radius R1 of about 0.0130 inches and a major radius R2 of about 0.0175 inches, thus providing the imaging lumen 32 with a distance across its minor axis of about 0.026 inches, a distance across its major axis of about 0.035 inches, an area of about 0.0007147 in², and a perimeter of about 0.09634 inches. The lumens may be sized such that the thickness T of the catheter wall is at least 0.003 inches at all locations of the catheter shaft 12. In other instances, the lumens may be sized such that the thickness T of the catheter wall is at least 0.002 inches, at least 0.0024 inches, at least 0.0026 inches, or at least 0.0028 inches, at all locations of the catheter shaft 112, for example.

As shown in FIG. 12, the imaging lumen 32 may have a cross-sectional area larger than the cross-sectional area of the guidewire lumen 24. However, other configurations are contemplated.

As shown in FIG. 12, the catheter shaft 112 may be constructed with the guidewire lumen 24 positioned entirely on one side of a plane Y parallel to and passing through the central longitudinal axis X of the catheter shaft 112. The imaging lumen 32 may be positioned such that the central longitudinal axis X of the catheter shaft 112 is within the imaging lumen 32 with a majority of the cross-sectional area of the imaging lumen 32 positioned on the opposite side of the plane Y parallel to and passing through the central longitudinal axis X.

Furthermore, as shown in FIG. 12, the catheter shaft 112 may be constructed such that a plane Z extending parallel to and passing through the central longitudinal axis X passes through the center of each of the guidewire lumen 24 and the imaging lumen 32. In some instances, the plane Z may be perpendicular to the plane Y.

The catheter shaft 112 may include a reinforcement structure 162, such as a braid or coil, to facilitate torque transmission of the catheter shaft such that the lateral opening may be rotationally oriented toward the true lumen of the blood vessel distal of the occlusion. For example, the catheter shaft 112 may include an extruded tubular member 160 defining the guidewire lumen 24 and the imaging lumen 32. A reinforcement structure 162, such as a braid or coil, may be disposed around (i.e., surround) the extruded tubular member 160, and an outer layer 164 may surround the reinforcement structure 162. As such, the catheter 110 may not include a balloon for rotational orientation.

The catheter 110 may be advanced into a subintimal space similar to that describe above regarding the catheter 10. The imaging lumen 32 is configured to receive an IVUS imaging device therein (as discussed above). The IVUS imaging device is configured to provide an ultrasound image of the blood vessel to confirm the rotational orientation of the lateral opening 150 in the subintimal space and/or the trajectory of a re-entry device to be advanced from the lateral opening 150 toward the true lumen. For example, one the orientation of the lateral opening 150 is confirmed with the IVUS imaging device, a reentry device may be advanced through the guidewire lumen 24 and out the lateral opening 150 to reenter the true lumen distal of the occlusion.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A recanalization catheter for facilitating reentry into a lumen of a vessel having an occlusion therein from a subintimal space in a wall of the vessel, the recanalization catheter comprising:
a catheter shaft extending distally from a hub, the catheter shaft defining a guidewire lumen extending therethrough;
first and second inflatable balloon members disposed on a distal end region of the catheter shaft, the catheter shaft including an inflation lumen in fluid communication with the first and second inflatable balloon members;
a lateral opening in communication with the guidewire lumen, wherein the lateral opening opens to an exterior of the catheter shaft between the first and second inflatable balloon members on a first side of the catheter shaft; and
an imaging lumen extending through the catheter shaft configured to receive an IVUS imaging device therein.

2. The recanalization catheter of claim 1, wherein the inflation lumen includes a first inflation lumen in fluid communication with the first inflatable balloon member and a second inflation lumen in fluid communication with the second inflatable balloon member.

3. The recanalization catheter of claim 2, wherein in a proximal portion of the catheter shaft a central longitudinal axis of the catheter shaft passes through the imaging lumen, and an entirety of each of the first inflation lumen, the second inflation lumen, and the guidewire lumen is positioned on a first side of a first imaginary plane extending parallel to and passing through the central longitudinal axis.

4. The recanalization catheter of claim 3, wherein in a distal portion of the catheter shaft, the imaging lumen and the guidewire lumen are positioned between the first and second inflation lumens such that a second imaginary plane extending parallel to and passing through the central longitudinal axis passes through each of the first inflation lumen, the second inflation lumen, the imaging lumen, and the guidewire lumen.

5. The recanalization catheter of claim 1, wherein a proximal portion of the catheter shaft has a circular cross-sectional shape and a distal portion of the catheter shaft has a generally rectangular cross-sectional shape.

6. The recanalization catheter of claim 1, wherein the first and second inflatable balloon member are configured to be inflated laterally from opposite sides of the catheter shaft along a third imaginary plane, wherein the third imaginary plane passes through each of the imaging lumen and the guidewire lumen.

7. The recanalization catheter of claim 1, further comprising an IVUS imaging device positionable within the imaging lumen to position an IVUS transducer distal of the first lateral opening.

8. The recanalization catheter of claim 1, wherein an outer diameter of the catheter shaft is 0.059 inches or less, the guidewire lumen has a diameter of about 0.017 inches, and a minimum distance across the imaging lumen through a center axis of the imaging lumen is 0.033 inches or more.

9. The recanalization catheter of claim 1, wherein an outer diameter of the catheter shaft is 0.052 inches or less, the guidewire lumen has a diameter of about 0.017 inches, and a minimum distance across the imaging lumen through a center axis of the imaging lumen is 0.026 inches or more.

10. The recanalization catheter of claim 9, wherein a wall thickness of the catheter shaft at all locations along the catheter shaft is at least 0.003 inches.

11. A recanalization catheter for facilitating reentry into a lumen of a vessel having an occlusion therein from a subintimal space in a wall of the vessel, the recanalization catheter comprising:
a catheter shaft extending distally from a hub,
an expandable balloon disposed on a distal end region of the catheter shaft;
wherein the catheter shaft is an extruded tubular member defining:
a guidewire lumen extending therethrough;
an inflation lumen in fluid communication with an interior of the expandable balloon; and
an imaging lumen extending through the catheter shaft configured to receive an IVUS imaging device therein.

12. The recanalization catheter of claim 11, further comprising an IVUS imaging device positional within the imaging lumen such that an IVUS transducer of the IVUS imaging device is located distal of the expandable balloon.

13. The recanalization catheter of claim 11, further comprising a lateral opening in communication with the guidewire lumen, wherein the lateral opening opens to an exterior of the catheter shaft between a proximal end of the expandable balloon and a distal end of the expandable balloon.

14. The recanalization catheter of claim 11, wherein the inflation lumen includes a first inflation lumen in fluid communication with a first inflatable balloon member of the expandable balloon and a second inflation lumen in fluid communication with a second inflatable balloon member of the expandable balloon;
wherein in a proximal portion of the catheter shaft a central longitudinal axis of the catheter shaft passes through the imaging lumen, and an entirety of each of the first inflation lumen, the second inflation lumen, and the guidewire lumen is positioned on a first side of a first imaginary plane extending parallel to and passing through the central longitudinal axis.

15. The recanalization catheter of claim 14, wherein in a distal portion of the catheter shaft, the imaging lumen and the guidewire lumen are positioned between the first and second inflation lumens such that a second imaginary plane extending parallel to and passing through the central longitudinal axis passes through each of the first inflation lumen, the second inflation lumen, the imaging lumen, and the guidewire lumen.

16. A recanalization catheter for facilitating reentry into a lumen of a vessel having an occlusion therein from a subintimal space in a wall of the vessel, the recanalization catheter comprising:
a catheter shaft extending distally from a hub,
an expandable balloon disposed on a distal end region of the catheter shaft, the expandable balloon including a first inflatable balloon member on a first lateral side of the catheter shaft and a second inflatable balloon member on a second lateral side of the catheter shaft opposite the first lateral side;
wherein the catheter shaft includes an extruded proximal tubular member having a circular cross-sectional shape and an extruded distal tubular member having a generally rectangular cross-sectional shape; the catheter shaft defining:
a guidewire lumen extending through the extruded proximal tubular member and the extruded distal tubular member;
a first inflation lumen extending through the extruded proximal tubular member and the extruded distal tubular member, the first inflation lumen in fluid communication with an interior of the first inflatable balloon member;
a second inflation lumen extending through the extruded proximal tubular member and the extruded distal tubular member, the second inflation lumen in fluid communication with an interior of the second inflatable balloon member; and
an imaging lumen extending through the extruded proximal tubular member and the extruded distal tubular member, the imaging lumen configured to receive an IVUS imaging device therein.

17. The recanalization catheter of claim 16, further comprising a lateral opening in communication with the guidewire lumen, wherein the lateral opening opens to an exterior of the catheter shaft between the first and second inflatable balloon members.

18. The recanalization catheter of claim 17, further comprising an IVUS imaging device positional within the imaging lumen such that an IVUS transducer of the IVUS imaging device is located distal of the lateral opening, preferably distal of the expandable balloon.

19. The recanalization catheter of claim 16, wherein throughout the extruded proximal tubular member a central longitudinal axis of the catheter shaft passes through the imaging lumen, and an entirety of each of the first inflation lumen, the second inflation lumen, and the guidewire lumen is positioned on a first side of a first imaginary plane extending parallel to and passing through the central longitudinal axis.

20. The recanalization catheter of claim 19, wherein throughout the extruded distal tubular member, the imaging lumen and the guidewire lumen are positioned between the first and second inflation lumens such that a second imaginary plane extending parallel to and passing through the central longitudinal axis passes through each of the first inflation lumen, the second inflation lumen, the imaging lumen, and the guidewire lumen.
